SEQUENCE LISTING (2 sequences)

SEQ ID NO: 1 - protein, 181 aa, Artificial sequence
  Synthetic polypeptide
MGPWNKDEISTTDAIFVQRDLKLVQGFMPHFFRLFRSTVKQVDFSEVERARFIINDWVKTHTKGMISNLLGKGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWK

SEQ ID NO: 2 - protein, 181 aa, Artificial sequence
  Synthetic polypeptide
MGPWNKDEISTADAIFVQRDLKLVQGFMPYFFRLFRTTVKQVDFSEMDRARFIINDWVKRHTKGMINDLLGQGAVDQLTRLVLVNALYFNGQWKTPFPEKSTHHRLFHKSDGSTVSVPMMAQTNKFNYTEFSTPDGHYYDILELPYHGNTLSMFIAAPYEKEVPLSALTSILDAQLISQWK

(19) United States
(12) Patent Application Publication
(10) Pub. No.: US 2016/0199466 A1
(43) Pub. Date: Jul. 14, 2016

(54) Methods For Treating Tissue Fibrosis

(71) Applicant: TRUSTEES OF DARTMOUTH COLLEGE, Hanover, NH (US)

(72) Inventor: Mary Jo Mulligan-Kehoe, Huntington Beach, CA (US)

(21) Appl. No.: 14/913,357

(22) PCT Filed: Jul. 29, 2014
(86) PCT No.: PCT/US2014/048604

§ 371 Date: Feb. 20, 2016

Related U.S. Application Data
(60) Provisional application No. 61/867,801, filed on Aug. 20, 2013.

(51) Int. Cl. A61K 38/57

(52) U.S. Cl. CPC A61K 38/57

(57) ABSTRACT

The present invention includes methods for reducing tissue damage in patients undergoing radiation treatment or with idiopathic pulmonary fibrosis or scleroderma by inhibiting PAI-1 activity in the tissue with a truncated PAI-1 agent, rPAI-1₂₃.

This application claims the benefit of priority from International Patent Application No. PCT/US2014/048604, filed Jul. 29, 2014, and U.S. Provisional Patent Application Ser. No. 61/867,801, filed Aug. 20, 2013, the contents of which are incorporated herein by reference in their entirety.

### BACKGROUND

Radiation therapy (RT) is commonly used in combination with chemotherapy for treatment of cancer. Although radiation treatment is effective, its curative dose is limited due to the risk of fibrosis in normal soft tissue within the field of radiation. Fibrosis, an end-stage pathological effect of radiation therapy, can lead to impaired quality of life, organ failure and death. There is a need for therapeutics that can protect tissue from radiation-induced damage, thus enabling the use of more effective, higher dose radiation therapy in patients while minimizing serious adverse effects associated with fibrotic tissue damage.

During normal tissue repair, damaged cells are replaced with the same type of cells. Fibrosis, a fibroproliferative disease, can occur under abnormal or pathogenic conditions. In this situation, connective tissue replaces parenchymal tissue in an uncontrolled manner to result in significant extracellular matrix (ECM) deposition and permanent scarring (Schuppan et al. 2001. Semin. Liver Dis. 21:351-372). Fibrosis is caused by an increased total amount of collagen that is a result of excess collagen synthesis relative to its rate of degradation (Pardo and Selman. 2006. Proc. Am. Thorac. Soc. 3:383-388).

Tissue repair during normal wound healing requires initiation of inflammation. Chronic inflammation and repair can stimulate excess ECM accumulation that leads to permanent fibrotic scar (Ghosh and Vaughan. 2012. J. Cell. Physiol. 227:493-507). Prolonged inflammation can also stimulate over-expression and/or activation of cytokines such as fibroblast growth factor-2 (FGF2) and transforming growth factor-beta (TGF-β). It is thought that macrophage-derived TGF-β stimulates fibrosis through activation of resident mesenchymal cells that differentiate into collagen producing myofibroblsts (Wynn, T. A. 2008. J. Pathol. 214:199-210).

TGF-β has been considered by some to be the “master switch” that stimulates a sequence of events culminating in radiation-induced fibrosis (Martin et al. 2000. J. Radiat. Oncol. Biol. Phys. 47:277-290; Roberts, A. B. 1995. Wound Repair Regen. 3:408-418). Its protein expression is elevated within 6 hours after radiation exposure, and continues to be elevated in fibrotic lesions for as long as 20 years post-radiation exposure (Martin et al. 2000. J. Radiat. Oncol. Biol. Phys. 47:277-290; Randall and Goggle. 1996. Int. J. Radiat. Biol. 70:351-360). However, others have identified TGF-β1-independent mechanisms which contribute to fibrosis in the lung and other tissues (Ashcroft et al. 1999. Nature Cell Biol. 1:260-266; Kaviratne et al. 2004. J Immunol. 173:4020-4029; Ma et al. 2003. Am. J. Pathol. 163:1261-1273).

TGF-β regulates transcription of numerous genes, including plasminogen activator inhibitor-1 (PAI-1; Eitzman et al. 1996. J. Clin. Invest. 97:232-237; Loskutoff et al. 2000. J. Clin. Invest. 106:1441-1443). PAI-1 is the primary inhibitor of the fibrinolytic pathway, thereby contributing to ECM accumulation if its levels are not tightly regulated (Ghosh and Vaughan. 2012. J. Cell. Physiol. 227:493-507). Mice that overexpress PAI-1 are sensitive to bleomycin-induced fibrosis, while those that lack the PAI-1 gene are resistant to fibrosis (Eitzman et al 1996. J. Clin. Invest. 97:232-237). When radiation and TGF-β treatment are combined, they have been shown to act synergistically to upregulate PAI-1 expression (Hageman et al. 2005. Clin. Cancer Res. 11:5956-5964), but radiation alone can stimulate PAI-1 expression. These data indicate that more than one pathway can stimulate PAI-1 effects on fibrosis.

A variety of studies have been performed examining the use of PAI-1 inhibitors, or modulating PAI-1 activity, to protect against radiation-induced tissue injury. Hageman et al. (2005. Clin. Cancer Res. 11:5956-5965) disclosed that PAI-1 has an important role in fibrosis that accompanies radiation therapy in cancer patients, including the cooperative roles of PAI-1 and TGF-β in inducing fibrosis in tissues. Specific PAI-1 inhibitors have been identified, e.g., PAI-749 (Gardell et al. 2007. Mol. Pharmacol. 72:897-906). Experiments have also been performed in PAI-1 knockout mice (Milliat et al. 2008. Am. J. Pathol. 172:691-701; Abderrahmani et al. 2009. Int. J. Radiation Oncology 74:942-948; Abderrahmani et al. 2012. PLoS ONE 7:e35740). These studies have shown that PAI-1 is essential for production of radiation-induced tissue injury in intestinal tissue (Milliat et al. 2008. Am. J. Pathol. 172:691-701), and that the level of PAI-1 is correlated with the severity of radiation-induced intestinal injury in PAI-1 knockout mice (Abderrahmani et al. 2009. Int. J. Radiation Oncology 74:942-948; Abderrahmani et al. 2012. PLoS ONE 7:e35740). In the 2009 publication, the authors also discussed the use of a PAI-1 inhibitor, PAI-039, which was shown to confer temporary protection against early lethality. In particular, while PAI-039 treatment limited the radiation-induced increase of connective tissue growth factor and PAI-1 at 2 weeks after irradiation, the inhibitor had no effect at 6 weeks.

U.S. Pat. No. 7,951,806, U.S. Pat. No. 5,415,479, U.S. Patent Application 2009/0124620, U.S. Patent Applications 2011/0112140, and U.S. Patent Application No. 2012/0022080 use of PAI-1 inhibitors to treat diseases, including radiation injury in tissues. The agents disclosed are chemically-synthesized agents and are not variants of PAI-1 itself.

U.S. Patent Application 2006/0084056 discloses that levels of PAI-1 in a cancer patient can be used to identify patients with poor outcomes. The application also discusses that levels of PAI-1 are related to the level of fibrosis in tissue.

U.S. Patent Application 2009/0227515 discloses peptides with activity to inhibit PAI-1 and their use to treat fibrotic damage in tissues, including radiation injury.

U.S. Pat. Nos. 7,241,446, 7,306,803, and 7,510,714 directed to methods for inhibiting angiogenesis. A variant of PAI-1 is taught in these patents, rPAI-1₂₃ (SEQ ID NO:1), which is a truncated form of PAI-1.

The truncated PAI-1 isoform, rPAI-1₂₃, is a potent angiogenesis inhibitor in vitro, ex vivo (Drinane et al. 2006. J. Biol. Chem. 281:33336-33344; Mulligan-Kehoe et al. 2002. J. Biol. Chem. 277:49077-49089; Mulligan-Kehoe et al. 2001. J. Biol. Chem. 276:8588-8596), and in a mouse model of atherosclerosis (Drinane et al. 2009. Circ. Res. 104:337-345; Mollmark et al. 2011. Circ. Res. 108:1419-1428; Mollmark et al. 2012. Arterioscl. Thromb. Vase. Biol. 32:2644-2651). Data showed that rPAI-1₂₃ blocks native PAI-1 in vascular tissue through a novel pathway that increases plasmin activity (Mollmark et al. 2011. Circ. Res. 108:1419-1428). The plasmin activity degrades key components of the ECM/basement membrane (BM) to result in loss of binding sites for FGF-2 (Mollmark et al. 2012. Arterioscl. Thromb. Vase. Biol. 32:2644-2651), a key factor in wound healing, angiogenesis and a potential fibrosis stimulator (Gridley et al. 2004. Int. J. Radiat. Oncol. Biol. Phys. 60:759-766; Masola et al. 2012. J. Biol. Chem. 287:1478-1488; Traub et al. 2012. Int. J. Colorect. Dis. 27:879-884).

### SUMMARY OF THE INVENTION

The present invention is a method of inhibiting PAI-1 activity in irradiated tissue comprising contacting irradiated tissue with an effective amount of a PAI-1 wherein the PAI-1 inhibitor comprises rPAI-1₂₃ (SEQ ID NO:1 or SEQ ID NO:2). In a preferred embodiment the tissue is lung tissue. The PAI-1 inhibitor can be administered either before or after irradiation.

Another object of the present invention is a method of reducing tissue fibrosis comprising contacting tissue with an effective amount of a PAI-1 inhibitor comprising SEQ ID NO: 1 or SEQ ID NO:2, so that tissue fibrosis is reduced. In one embodiment, the tissue fibrosis is induced by radiation. In certain embodiments, the tissue is lung tissue. In other embodiments, tissue fibrosis is associated with diseases such as idiopathic pulmonary fibrosis and scleroderma. Also contemplated is administration of the PAI-1 inhibitor in a pharmaceutically acceptable vehicle.

Yet another object of the present invention is a method of preventing or treating tissue fibrosis induced by radiation or fibrotic disease comprising contacting tissue with an effective amount of a PAI-1 inhibitor comprising SEQ ID NO: 1 or SEQ ID NO: 2 so that tissue fibrosis is inhibited. In some embodiments, the tissue is contacted before radiation treatment or after radiation treatment. In preferred embodiments the tissue is lung or skin tissue. In other embodiments, the fibrotic disease is idiopathic pulmonary fibrosis or scleroderma. Also contemplated is administration of the PAI-1 inhibitor in a pharmaceutically acceptable vehicle.

Another object of the present invention is a method of increasing the dose of radiation that can be administered to a patient undergoing radiation treatment for cancer comprising administering an effective amount of a PAI-1 inhibitor of SEQ ID NO:1 or SEQ ID NO: 2 before radiation treatment. In a preferred embodiment, the cancer being treated is lunch cancer.

### BRIEF DESCRIPTION OF THE DRAWINGS

FIG. 1 depicts the role of the recombinant PAI-1 of the present invention, rPAI-1₂₃, to enhance plasmin activity through interactions with plasminogen. In the figure, (A) shows the tissue plasminogen activator (tPA) cleaves plasminogen (Pig), which converts Plg to proteolytic plasmin; (B) shows that PAI-1 binds tPA to prevent tPA interaction with Plg thus inhibiting plasmin production; (C) shows that rPAI-1₂₃ binds Plg, which increases plasmin activity; and (D) shows that rPAI-1₂₃ to plasminogen inhibits PAI-1 activity by sequestering PAI-1 to a Plg complex thus limiting its ability to inhibit tPA.

FIG. 2 shows the results of in vivo experiments where rPAI-1₂₃ treatment reduced fibrosis in irradiated lung tissue of mice. C57BL/6NCr mice were exposed to 6 Gy×5 of radiation and treated with rPAI-1₂₃ or saline for 120 days. Lung tissue was harvested 22 weeks after radiation treatment. The graphs depict the results when collagen density was determined using imaging of stained areas. ** p<0.01.

FIG. 3 depicts results of in vivo experiments in mice where treatment with rPAI-1₂₃ inhibited leg shortening in irradiated limbs. C3H/HeN mice were irradiated at 2.61 Sixteen mice received 35 Gy to the right leg and treated with either rPAI-1₂₃ or saline. Hind leg contracture was measured at 90, 120, 150 and 180 days. Control mice were not irradiated but were treated with either rPAI-1₂₃ or saline. Results showed that at 180 days post-radiation, and 34 days after treatment withdrawal, rPAI-1₂₃ sustained inhibitory effects on leg contracture. *p>0.05.

### DETAILED DESCRIPTION OF THE INVENTION

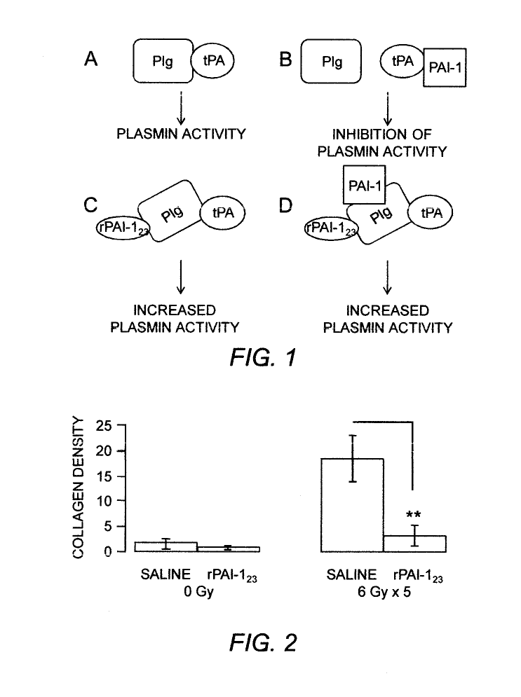

The present invention is directed to a novel truncated variant of PAI-1 which has activity to inhibit PAI-1 and prevent radiation-induced tissue damage, in particular tissue fibrosis. Thus, the present invention is a method for reducing radiation-induced tissue damage, in particular tissue fibrosis, using the PAI-1 inhibitor rPAI-1₂₃ (SEQ ID NO:1 or SEQ ID NO:2). The activity of the truncated PAI-1 variant has been demonstrated both in vitro and in vivo. The activity of rPAI-1₂₃ has been shown to involve a pathway as described in FIG. 1. As shown in FIG. 1, rPAI-1₂₃ enhances plasmin activity through interactions with plasminogen. It is known that PAI-1 binds tPA and prevents tPA from cleaving plasminogen and converting it to plasmin. rPAI-1₂₃ binds plasminogen, which increases plasmin activity. rPAI-1₂₃ bound to plasminogen inhibits PAI-1 activity by sequestering PAI-1 to a plasminogen complex thereby preventing inhibition of tPA activity. As is understood by one of skill in the art, a decrease in PAI-1 activity is an effect that would be beneficial clinically to reduce fibrosis associated with radiation exposure in organs such as lung.

The major limiting factor for determining the dose of radiation for the treatment of cancer is damage to normal tissue. As a result the treatment regimens are often below the curative dose. This is particularly the case for lungs, which are highly radiosensitive (Abratt et al. 2002. Lung Cancer 36:225-233; Abratt and Morgan. 2002. Lung Cancer 35:103-109; Movsas et al. 1997. Chest 111:1061-1076) and where fibrosis is the most common toxicity following radiation therapy (RT) of the lung (Morgan, G. W. and S. N., Breit. 1995. Int. J. Radiat. Oncol. Biol. Phys. 31:361-369; Peters, L. J. 1996. Cancer 77:2379-2385). The extent of radiation-induced fibrosis, and other toxicities, is dependent upon radiation dose, dose rate, fractionation schedule, and the total irradiated volume (Movsas et al. 1997. Chest 111:1061-1076). Lung complications associated with RT have become more prevalent due to more aggressive RT and increased use of RT in combination with chemotherapy (Abid et al. 2001. Curr. Opin. Oncol. 13:242-248). Studies have shown that patients receiving concurrent chemotherapy and radiation therapy for treatment of limited small cell lung carcinoma have improved survival. Follow up computed tomography (CT) studies after 1 year show that those who were irradiated with a dose greater than 30-35 Gy had an increased probability of lung fibrosis. If patients who received accelerated fractionation of twice daily are compared to those receiving a conventional single dose per day, there was a two-fold increase in probability of tissue fibrosis (Rosen et al. 2001. Radiology 221:614-622; Turrisi et al. 1999. New Engl. J. Med. 340:265-271; Geara et al. 1998. Int. J. Radiat. Oncol. Biol. Phys. 41:279-286). Therefore, there is a need for an inhibitor of fibrosis that would enable RT treatment of cancer within a curative radiation dose.

Studies in a mouse model of hypercholesterolemia have demonstrated that rPAI-1₂₃ protein modulates the functions of PAI-1 and FGF2, both of which play significant roles in wound healing and are known to contribute to fibrosis (Mollmark et al. 2012. Arterioscler. Thromb. Vase. Biol. 32:2644-2651; Mollmark et al. 2012. Circ. Res. 108:1419-1428). Mechanistic studies showed that rPAI-1₂₃ plasmin activity through a novel pathway that increases plasmin activity and blocks PAI-1 activity via a complex formed with plasminogen (FIG. 1). The elevated plasmin activity contributes to rPAI-1₂₃ anti-angiogenic activity by degrading key components of the basement membrane/extracellular matrix (BM/ECM) that are necessary for stability of angiogenic vessels. The altered matrix affects FGF2 pro-angiogenic activity by disrupting its interaction with porleean (Mollmark et al. 2012. Arterioscl. Thromb. Vase. Biol. 32:2644-2651). The combined studies clearly demonstrated that rPAI-1₂₃ activity can modify the plasminogen activator and angiogenesis pathways, which are intrinsic to normal wound healing process, but deleterious when uncontrolled.

With the earlier work focused on angiogenesis and a role for inhibition of PAI-1 with rPAI-1₂₃, investigations were extended to a different situation where activity of PAI-1 was operative, i.e., radiation-induced tissue damage. It has now been found that in lung tissue, radiation treatment accompanied by administration of rPAI-1₂₃ has the potential to allow delivery of a more aggressive radiation regimen to cancer patients, thus significantly improving survival time and eliminating the complications of fibrosis.

Experiments were performed to test the effect of rPAI-1₂₃ on radiation-induced lung damage in vivo. In a lung fibrosis model, ten week-old female C57BL/6NCr mice were restrained in a LUCITE jig that allowed irradiation of the thorax and shielded the remainder of the body with lead. Cohorts of mice were treated with 0 Gy (control) or 5×6 directed to the thoracic region, and followed until 16 (n=16 per dose). Separate cohorts of mice (n=16 per dose) were treated with 0 Gy (control) or 5×6 Gy directed to the thoracic region, and followed for 22 weeks. Eight mice from each cohort were treated with either vehicle, phosphate-buffered saline (PBS), or rPAI-1₂₃ (5 μg/kg/day) by intraperitoneal (IP) injection beginning 2 days prior to irradiation and continuing for 120 days. Additional mice were treated with no irradiation and either vehicle or rPAI-1₂₃. Lung tissue harvested 22 weeks after radiation was stained with Massons trichrome to detect collagen. As seen in FIG. 2, examination under the microscope showed that no appreciable collagen areas were seen in tissues of the control mice (0 Gy irradiation and treatment with either saline or rPAI-1₂₃). In contrast, lung tissue sections taken from mice irradiated with 6×5 Gy and treated with saline had significant areas of collagen staining (FIG. 2). Treatment with rPAI-1₂₃, a PAI-1 inhibitor, significantly reduced the collagen accumulation in lung tissue sections (FIG. 2; p<0.01 as compared to saline controls). These data demonstrated that rPAI-1₂₃ prevents and treats lung damage, specifically tissue fibrosis, in vivo.

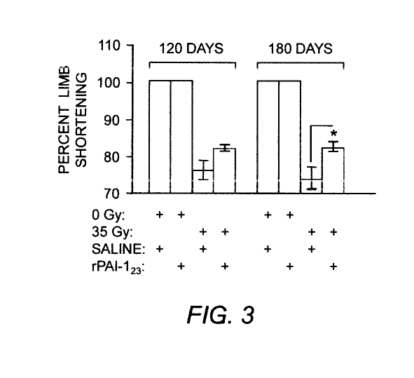

In another set of experiments, ten week-old female C3H/HeN mice were restrained in a LUCITE jig that allowed irradiation of the right hind leg and shielded the remainder of the body with lead. Radiation was delivered with an X-RAD 320 (Precision X-Ray, North Brandford, Conn.) with 2.0 mm Aluminum (Al) filtration (300 kv peak) at 2.61 Sixteen mice received 35 Gy to the right hind leg. Eight of these mice were injected IP with rPAI-1₂₃ (5 μg/kg/day), while eight other mice (controls) received saline via IP injection. Additional control mice were treated with no irradiation and either vehicle or rPAI-1₂₃ dose as the irradiated mice). Dosing was initiated two days prior to irradiation and continued for 120 days. Hind leg contracture was serially assessed at 90, 120, 150 and 180 days after irradiation by measuring extension of the irradiated and contralateral hind limbs under light anesthesia. Results showed that rPAI-1₂₃ treatment had a significant inhibitory effect on high dose radiation-induced fibrosis, an effect that was sustained for 34 days after withdrawal of rPAI-1₂₃ treatment (FIG. 3; *p<0.05). In addition, rPAI-1₂₃ (SEQ ID NO:1) was shown to be associated in vivo with a significant reduction in tissue fibrosis in lung, an effect commonly seen with high dose radiation exposure.

Thus, the present invention provides a method to affect activity of PAI-1, prevent and/or reduce fibrosis in tissue and, therefore, prevent and/or treat fibrosis associated with fibrotic disease or radiation-induced tissue damage in patients, in particular lung cancer patients. The present invention is a method of inhibiting PAI-1 activity in irradiated tissue by contacting irradiated tissue with an effective amount of a PAI-1 wherein the PAI-1 inhibitor is rPAI-1₂₃ (SEQ ID NO:1 or SEQ ID NO:2).

As used herein, rPAI-1₂₃ is a recombinant plasminogen activator inhibitor-1 isoform that lacks the reactive center loop domain, located at amino acids 320-351, and lacks at least a portion of the heparin-binding domain. The sequence of human and porcine rPAI-1₂₃ are provided as SEQ ID NO:1 and SEQ ID NO:2, respectively.

In a preferred embodiment the tissue is lung tissue. The PAI-1 inhibitor can be administered either before or after irradiation. In another embodiment, the present invention is a method of reducing tissue fibrosis associated with radiation treatment or fibrotic disease comprising contacting tissue with an effective amount of a PAI-1 inhibitor comprising SEQ ID NO:1 or SEQ ID NO:2, so that tissue fibrosis is reduced. In one embodiment, the tissue to be irradiated is contacted with an effective amount of a PAI-1 inhibitor comprising SEQ ID NO:1 or SEQ ID NO:2. In a preferred embodiment the tissue is lung tissue. In certain embodiments, the disease is idiopathic pulmonary fibrosis or scleroderma. Also contemplated is administration of the PAI-1 inhibitor in a pharmaceutically acceptable vehicle. Yet another embodiment of the present invention is a method of preventing or treating tissue fibrosis induced by radiation or fibrotic disease comprising contacting tissue with an effective amount of a PAI-1 inhibitor comprising SEQ ID NO:1 or SEQ ID NO: 2. In one embodiment, the tissue is contacted with a PAI-1 before radiation treatment or after radiation treatment so that tissue fibrosis is inhibited. In preferred embodiments the tissue is lung tissue. In another embodiment, the disease being prevented or treated is idiopathic pulmonary fibrosis or scleroderma. In accordance with this method, prevention or treatment refers the prophylactic or therapeutic administration of the PAI-1 so that fibrosis does not occur, fibrosis is reduced or inhibited, or further fibrosis does not occur (e.g., in a subject with fibrosis). Also contemplated is administration of the PAI-1 inhibitor in a pharmaceutically acceptable vehicle.

Finally, the present invention is a method of increasing the dose of radiation that can be administered to a patient undergoing radiation treatment for cancer comprising administering an effective amount of a PAI-1 of SEQ ID NO:1 or SEQ ID NO: 2 before radiation treatment. In a preferred embodiment the cancer being treated is lung cancer.

In the context of the present invention “an effective amount” of rPAI-1₂₃ is a dose that significantly inhibits activity of PAI-1 in tissue, thereby inhibiting fibrosis. It is contemplated that one of skill in the art would use routine experimentation to optimize the dosing regimen for rPAI-1₂₃ in terms of both the magnitude of the dose and the frequency of the treatment, i.e., daily, twice a day, etc. Additionally, one of skill would be able to determine how long to continue dosing with rPAI-1₂₃ based on the length of time that radiation treatment would continue and/or the severity of disease. Doses of from 0.1 to 2 μg/kg/day are envisioned as being a dose range for treatment of animals, including humans (e.g., having an average weight of 60 kg).

When used in the methods of this invention, rPAI-1₂₃ administered to a subject in need of treatment in an amount that effectively inhibits PAI-1 activity in tissue (e.g., radiation-treated tissue or diseased tissue). The level of PAI-1 inhibition could range anywhere from 10% to 100%, with a preferred level of inhibition of at least 25%. In the context of this invention, a patient or subject can be any mammal including human, companion animals (e.g., dogs or cats), livestock (e.g., cows, sheep, pigs, or horses), laboratory animal (e.g., rabbits), or zoological animals (e.g., monkeys). In particular embodiments, the subject is a human.

rPAI-1₂₃ is also useful in a method of treatment of cancer wherein the combination of rPAI-1₂₃ in the treatment regimen would allow for higher doses of radiation to be given to the patient in order to treat the cancer. Therefore, the present invention is a method of increasing the dose of radiation that can be administered to a patient undergoing radiation treatment for cancer comprising administering an effective amount of a PAI-1 inhibitor of SEQ ID NO:1 or SEQ ID NO:2 before radiation treatment. In a preferred embodiment the cancer being treated is lung cancer. The method can involve treatment with the PAI-1 before or during radiation treatment so that the dose of radiation used can be increased and the cancer is treated. Although lung cancer is a specific cancer contemplated for treatment using radiation therapy and rPAI-1₂₃, other forms of cancer treated with radiation are also contemplated to be amenable to rPAI-1₂₃ treatment. Examples of such cancers include, but not limited to, any cancer where high doses of radiation (i.e., doses greater than 30Gy) are employed, e.g., pancreatic cancer, rectal cancer, and nasopharyngeal cancer. Use of the instant invention allows for use of higher doses of radiation without the risk of fibrosis.

When used in therapeutic applications, rPAI-1₂₃ will have the therapeutic benefit of decreasing, reducing or ameliorating the signs or symptoms of tissue fibrosis (e.g., radiation-induced tissue fibrosis) as compared to subjects not receiving treatment with rPAI-1₂₃. It is contemplated that treatment with rPAI-1₂₃ would be given before RT, or immediately after RT, and continued through the regimen.

For therapeutic use, rPAI-1₂₃ can be formulated with a pharmaceutically acceptable carrier at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier, composition or vehicle, such as a liquid or solid filler, diluent, excipient, or solvent encapsulating material, is involved in carrying or transporting the subject agent from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Compositions of the present invention can be administered parenterally (for example, by intravenous, intraperitoneal, subcutaneous or intramuscular injection), topically, orally, intranasally, intravaginally, or rectally, according to standard medical practices.

The selected dosage level of rPAI-1₂₃ will depend upon a variety of factors including the route of administration, the time of administration, the duration of the treatment, other drugs, compounds and/or materials used in combination, the age, sex, weight, condition, general health and prior medical history of the patient being treated, and other factors well-known in the medical arts.

It is also contemplated that rPAI-1₂₃ can be used in the methods of the present invention either alone or in combination with other agents commonly used to treat radiation-induced tissue damage in patients. Such agents would include but not be limited to other PAI-1 inhibitors. One of skill in the art would choose which agents to use in combination based on their clinical experience with such agents, using doses approved for use in humans per the labeling for the drug products as marketed.

A physician having ordinary skill in the art can readily determine and prescribe the effective amount of the pharmaceutical composition required based upon the administration of similar compounds or experimental determination. For example, the physician could start doses of an agent at levels lower than that required in order to achieve the desired therapeutic effect and gradually increase the dosage until the desired effect is achieved. This is considered to be within the skill of the artisan and one can review the existing literature on a specific agent or similar agents to determine optimal dosing.

The following non-limiting examples are provided to further illustrate the present invention.

### Example 1

### Clinical Trial

Patients diagnosed with lung cancer are recruited for a clinical trial based on their need for high dose (>30Gy) radiation therapy. Standard clinical trial methods are applied using methods known to those of skill in the art. Blood samples are taken before first dosing with rPAI-1₂₃ in order to determine PAI-1 activity levels before initiation of treatment (baseline). Patients are treated two days before initiation of radiation therapy with an effective dose of rPAI-1₂₃ (i.e., 0.1 to 2.0 μg/kg/day; intravenous dosing). One group of patients receives a formulation vehicle injection (untreated control) throughout the study. Subsequently, radiation treatment is initiated. At one time point during radiation treatment, and at the end of the radiation treatment, blood samples are again drawn for monitoring PAI-1 activity levels. Although changes in PAI-1 activity levels are used as one measure of drug efficacy, the primary efficacy endpoint for the study is absolute change forced vital capacity (FVC), where treatment with rPAI-1₂₃ is expected to prevent or decrease declines in FVC that are associated with lung fibrosis. Other efficacy endpoints that can be monitored may include total lung capacity, total lung diffusion capacity for carbon monoxide, 6-minute walk distance, quality of life, and survival.

What is claimed is:

1. A method of inhibiting PAI-1 activity in irradiated tissue comprising contacting irradiated tissue with an effective amount of a PAI-1 inhibitor, wherein the PAI-1 comprises SEQ ID NO:1 or SEQ ID NO:2.

2. The method of claim 1, wherein said irradiated tissue is lung tissue.

3. The method of claim 1, wherein the irradiated tissue is contacted with the PAI-1 inhibitor before it is irradiated.

4. A method of reducing tissue fibrosis comprising contacting tissue with an effective amount of a PAI-1 comprising SEQ ID NO:1 or SEQ ID NO:2, so that tissue fibrosis is reduced.

5. The method of claim 4, wherein the tissue fibrosis is induced by radiation.

6. The method of claim 4, wherein the tissue fibrosis is from idiopathic pulmonary fibrosis or scleroderma.

7. The method of claim 4, wherein the tissue is lung tissue.

8. The method of claim 4, wherein the PAI-1 inhibitor is administered in a pharmaceutically acceptable vehicle.

9. A method of preventing or treating tissue fibrosis induced by radiation or a fibrotic disease comprising contacting tissue with an effective amount of a PAI-1 comprising SEQ ID NO:1 or SEQ ID NO:2 so that tissue fibrosis is inhibited.

10. The method of claim 9, wherein the tissue is contacted before radiation treatment or after radiation treatment.

11. The method of claim 9, wherein the fibrotic disease is idiopathic pulmonary fibrosis or scleroderma.

12. The method of claim 9, wherein the tissue is lung tissue.

13. The method of claim 9, wherein the PAI-1 inhibitor is administered in a pharmaceutically acceptable vehicle.

14. A method of increasing the dose of radiation that can be administered to a patient undergoing radiation treatment for cancer comprising administering an effective amount of a PAI-1 inhibitor of SEQ ID NO:1 or SEQ ID NO:2 radiation treatment.

15. The method of claim 14, wherein the cancer is lung cancer.
